(19) United States
(12) Patent Application Publication
(10) Pub. No.: US 2023/0322930 A1
(43) Pub. Date: Oct. 12, 2023

(54) USE OF AN ANTI-PD-1 ANTIBODY AND A CYTOTOXIC ANTICANCER DRUG IN TREATMENT OF NON-SMALL CELL LUNG CANCER

(71) Applicant: SHANGHAI JUNSHI BIOSCIENCES CO., LTD., SHANGHAI (CN)

(72) Inventors: Sheng Yao, Shanghai (CN); Hui Feng, Shanghai (CN)

(21) Appl. No.: 18/042,756

(22) PCT Filed: Aug. 27, 2021
(86) PCT No.: PCT/CN2021/114982

§ 371 Date: Feb. 23, 2023

(30) Foreign Application Priority Data
Aug. 28, 2020 (CN) 202010883912.3
Aug. 12, 2021 (CN) 202110925375.9

(51) Int. Cl. C07K 16/28; A61K 31/519; A61K 31/555; A61P 35/00

(52) U.S. Cl. CPC C07K 16/2827; A61K 31/519; A61K 2039/545; A61P 35/00; A61K 31/555

(57) ABSTRACT

Provided are a combination of an anti-PD-1 antibody or an antigen binding fragment of the anti-PD-1 antibody and a cytotoxic anticancer drug, and the use of the combination in the preparation of a medicine for treating non-small cell lung cancer. Specifically, Provided are a combination of an anti-PD-1 antibody or an antigen-binding fragment of the anti-PD-1 antibody, an anti-folate metabolism anticancer drug, and a platinum anticancer drug, and the use of the combination in the preparation of a medicine for treating non-small cell lung cancer that has failed EGFR-TKI treatment.

### TECHNICAL FIELD

The present invention relates to a combination of an anti-PD-1 antibody or an antigen binding fragment of the anti-PD-1 antibody and a cytotoxic anticancer drug, and the use of the combination in the preparation of a medicine for treating non-small cell lung cancer. Specifically, the present invention relates to a combination of an anti-PD-1 antibody or an antigen-binding fragment of the anti-PD-1 antibody, an anti-folate metabolism anticancer drug, and a platinum anticancer drug, and the use of the combination in the preparation of a medicine for treating non-small cell lung cancer that has failed EGFR-TKI treatment.

### BACKGROUND ART

In the past decade, the treatment prospects of patients with advanced non-small cell lung cancer (NSCLC) with EGFR sensitization mutation have undergone tremendous changes. The median progression-free survival (mPFS) of first-line EGFR TKIs, such as erlotinib, gefitinib, icotinib, afatinib, and osimertinib, is 19 months. However, after the failure of the first-line TKI treatment, the mPFS of the second or subsequent treatment is significantly reduced. For T790M resistant mutation in about 60% of patients who receive osimertinib treatment, most patients will eventually progress and receive subsequent chemotherapy, but the clinical efficacy is limited (mOS is 12 months). Therefore, there is an urgent need for new strategies to further improve the prognosis of this population after the failure of EGFR-TKIs.

Programmed death receptor 1 (PD-1) plays an important role in immune regulation and maintenance of peripheral tolerance. PD-1 is mainly expressed in activated T cells and B cells. The function of PD-1 is to inhibit the activation of lymphocytes and this is a normal peripheral tissue tolerance mechanism of the immune system to prevent overactive immune response. However, activated T cells that infiltrate the tumor microenvironment highly express PD-1 molecules, and the inflammatory factors secreted by activated white blood cells will induce tumor cells to highly express PD-1 ligands, PD-L1 and PD-L2, resulting that the PD-1 pathway of activated T cells in the tumor microenvironment is continuously activated, and the function of T cells is inhibited and T cells are unable to kill tumor cells. Therapeutic PD-1 antibody can block this pathway, partially restore the function of T cells, so that activated T cells can continue to kill tumor cells.

Currently, immunotherapy has completely changed our treatment for advanced/metastatic NSCLC without EGFR mutations. Immune checkpoint inhibitors (ICIs) against the PD-1/PD-L1 pathway, such as nivolumab, pembrolizumab, and atezolizumab, have been included in the second-line treatment criteria for patients with advanced NSCLC. In addition, monotherapy with pembrolizumab or atezolizumab is also the first-line choice for advanced NSCLC patients with high PD-L1 expression in tumor biopsy. However, compared with patients receiving criteria chemotherapy, patients with EGFR mutations who receive monotherapy of PD-L1 or PD-1 inhibitor after TKI failure do not show a substantial survival benefit. In addition, clinical trials of the combined use of osimertinib and an PD-L1 antibody have led to safety issues such as the high incidence of interstitial pneumonia. Therefore, for NSCLC patients with EGFR mutations after TKI treatment failure, better alternative strategies are still needed.

### SUMMARY OF THE INVENTION

The present invention provides the use of a combination of an anti-PD-1 antibody or an antigen binding fragment of the anti-PD-1 antibody and a cytotoxic anticancer drug in the preparation of a medicine for treating non-small cell lung cancer.

In one or more embodiments, the non-small cell lung cancer of the present invention is a non-small cell lung cancer that has failed EGFR-TKI treatment; preferably, the non-small cell lung cancer is advanced or recurrent non-small cell lung cancer that has failed EGFR-TKI treatment and has EGFR mutations.

In one or more embodiments, the EGFR mutations of the present invention are selected from exon 19 deletion and L858R mutation in exon 21; preferably, the L858R mutation in exon 21.

In one or more embodiments, the non-small cell lung cancer of the present invention is a non-small cell lung cancer with PD-L1 expression ≥1% in immunohistochemical staining analysis of tumor tissue sections; preferably, a non-small cell lung cancer with PD-L1 expression 10% in the immunohistochemical staining analysis of tumor tissue sections.

In one or more embodiments, the anti-PD-1 antibody or the antigen binding fragment of the anti-PD-1 antibody of the present invention comprises light chain complementarity determining regions with the amino acid sequences shown in SEQ ID NOs: 1, 2 and 3, and heavy chain complementarity determining regions with the amino acid sequences shown in SEQ ID NOs: 4, 5, and 6.

In one or more embodiments, the anti-PD-1 antibody or the antigen binding fragment of the anti-PD-1 antibody of the present invention comprises a light chain variable region with the amino acid sequence shown in SEQ ID NO: 7, and a heavy chain variable region with the amino acid sequence shown in SEQ ID NO: 8.

In one or more embodiments, the anti-PD-1 antibody of the present invention comprises a light chain with the amino acid sequence shown in SEQ ID NO: 9 and a heavy chain with the amino acid sequence shown in SEQ ID NO: 10.

In one or more embodiments, the anti-PD-1 antibody of the present invention is selected from one or more of nivolumab, pembrolizumab, toripalimab, sintilimab, camrelizumab, tislelizumab, and cemiplimab; preferably, toripalimab.

In one or more embodiments, the cytotoxic anticancer drug of the present invention is an anti-folate metabolism anticancer drug and/or a platinum anticancer drug.

In one or more embodiments, the anti-folate metabolism anticancer drug of the present invention is selected from methotrexate or pemetrexed; preferably, pemetrexed.

In one or more embodiments, the platinum anticancer drug of the present invention is selected from cisplatin, carboplatin and oxaliplatin; preferably, carboplatin.

In one or more embodiments, the combination is a combination of an anti-PD-1 antibody or an antigen binding fragment of the anti-PD-1 antibody, an anti-folate metabolism anticancer drug and a platinum anticancer drug.

In one or more embodiments, the combination is a combination of an anti-PD-1 antibody or an antigen binding fragment of the anti-PD-1 antibody and an anti-folate metabolism anticancer drug.

In one or more embodiments, the combination is a combination of toripalimab and pemetrexed.

In one or more embodiments, the combination is a combination of toripalimab, pemetrexed, and carboplatin.

In one or more embodiments, in the use of the present invention,

(I) the single administration dose of the anti-PD-1 antibody or the antigen binding fragment of the anti-PD-1 antibody is about 0.1 mg/kg to about 10.0 mg/kg of individual body weight, for example, about 0.1 mg/kg, about 0.3 mg/kg, about 1 mg/kg, about 2 mg/kg, about 3 mg/kg, about 5 mg/kg, or 10 mg/kg of individual body weight, or selected from a fixed dose of about 120 mg to about 480 mg, such as a fixed dose of 120 mg, 240 mg, 360 mg or 480 mg, preferably a fixed dose of 240 mg and 360 mg;

(II) the cytotoxic anticancer drug is an anti-folate metabolism anticancer drug, wherein the single administration dose of the anti-folate metabolism anticancer drug is selected from about 200 mg/m² to about 800 mg/m², preferably 400 mg/m², 500 mg/m² or 600 mg/m²; and/or, the cytotoxic anticancer drug is a platinum anticancer drug, wherein the single administration dose of the platinum anticancer drug is selected from about 200 mg/m² to about 800 mg/m², preferably 400 mg/m², 500 mg/m² or 600 mg/m².

In one or more embodiments, in the use of the present invention,

(I) the dosing frequency of the anti-PD-1 antibody or the antigen binding fragment of the anti-PD-1 antibody is about once a week, once every two weeks, once every three weeks, once every four weeks, or once a month, preferably once every three weeks;

(II) the dosing frequency of the anti-folate metabolism anticancer drug is about once a week, once every two weeks, once every three weeks, once every four weeks or once a month, preferably once every three weeks;

(III) the dosing frequency of the platinum anticancer drug is about once a week, once every two weeks, once every three weeks, once every four weeks, or once a month, preferably once every three weeks.

In one or more embodiments, in the use of the present invention,

(I) the anti-PD-1 antibody or the antigen binding fragment of the anti-PD-1 antibody has an administration dose of a fixed dose of 240 mg or 360 mg and administered once every three weeks;

(II) the anti-folate metabolism anticancer drug has an administration dose of 500 mg/m² and administered once every three weeks;

(III) the platinum anticancer drug has an administration dose of 500 mg/m² and administered once every three weeks.

In one or more embodiments, in the use of the present invention, the dosing period of the anti-PD-1 antibody or the antigen binding fragment of the anti-PD-1 antibody, the anti-folate metabolism anticancer drug and/or the platinum anticancer drug may be one week, two weeks, three weeks, one month, two months, three months, four months, five months, six months or longer, optionally, each dosing period can be the same or different, and the interval between each dosing period can be the same or different.

In one or more embodiments, in the use of the present invention, the anti-PD-1 antibody or the antigen binding fragment of the anti-PD-1 antibody, the anti-folate metabolism anticancer drug and the platinum anticancer drug are administered in a liquid dosage form, for example an injection, through parenteral route, for example, intravenous infusion.

In yet another aspect, the present invention provides a pharmaceutical composition comprising the anti-PD-1 antibody or the antigen binding fragment of the anti-PD-1 antibody and the cytotoxic anticancer drug of the present invention, wherein the cytotoxic anticancer drug is an anti-folate metabolism anticancer drug and/or a platinum anticancer drug.

In one or more embodiments, in the pharmaceutical composition of the present invention,

the anti-PD-1 antibody or the antigen binding fragment of the anti-PD-1 antibody of the present invention comprises light chain complementarity determining regions with the amino acid sequences shown in SEQ ID NOs: 1, 2 and 3, and heavy chain complementarity determining regions with the amino acid sequences shown in SEQ ID NOs: 4, 5, and 6; preferably, the anti-PD-1 antibody or the antigen binding fragment of the anti-PD-1 antibody comprises a light chain variable region with the amino acid sequence shown in SEQ ID NO: 7, and a heavy chain variable region with the amino acid sequence shown in SEQ ID NO: 8; further preferably, the anti-PD-1 antibody comprises a light chain with the amino acid sequence shown in SEQ ID NO: 9 and a heavy chain with the amino acid sequence shown in SEQ ID NO: 10; preferably, the anti-PD-1 antibody is toripalimab; the anti-folate metabolism anticancer drug is selected from methotrexate or pemetrexed; preferably, pemetrexed; the platinum anticancer drug is selected from cisplatin, carboplatin and oxaliplatin; preferably, carboplatin.

In one or more embodiments, the anticancer active ingredients in the pharmaceutical composition are toripalimab and pemetrexed.

In one or more embodiments, the anticancer active ingredients in the pharmaceutical composition are toripalimab, pemetrexed and carboplatin.

In yet another aspect, the present invention provides a method for preventing or treating non-small cell lung cancer, wherein the method comprises the combined administration of an anti-PD-1 antibody or an antigen binding fragment of the anti-PD-1 antibody and a cytotoxic anticancer drug, or the pharmaceutical composition according to the present invention to an individual in need, wherein the cytotoxic anticancer drug is an anti-folate metabolism anticancer drug and/or a platinum anticancer drug; preferably, the anti-PD-1 antibody or the antigen binding fragment of the anti-PD-1 antibody is according to any of the embodiments herein; preferably, the cytotoxic anticancer drug is according to any of the embodiments herein.

In one or more embodiments, in the method of the present invention, the anti-PD-1 antibody or the antigen binding fragment of the anti-PD-1 antibody of the present invention comprises light chain complementarity determining regions with the amino acid sequences shown in SEQ ID NOs: 1, 2 and 3, and heavy chain complementarity determining regions with the amino acid sequences shown in SEQ ID NOs: 4, 5, and 6; preferably, the anti-PD-1 antibody or the antigen binding fragment of the anti-PD-1 antibody comprises a light chain variable region with the amino acid sequence shown in SEQ ID NO: 7, and a heavy chain variable region with the amino acid sequence shown in SEQ ID NO: 8; further preferably, the anti-PD-1 antibody comprises a light chain with the amino acid sequence shown in SEQ ID NO: 9 and a heavy chain with the amino acid sequence shown in SEQ ID NO: 10; preferably, the anti-PD-1 antibody is toripalimab; the anti-folate metabolism anticancer drug is selected from methotrexate or pemetrexed, preferably pemetrexed; and the platinum anticancer drug is selected from cisplatin, carboplatin and oxaliplatin, preferably carboplatin.

In one or more embodiments, in the method of the present invention, the single administration dose of the anti-PD-1 antibody or the antigen binding fragment of the anti-PD-1 antibody is about 0.1 mg/kg to about 10.0 mg/kg of individual body weight, for example, about 0.1 mg/kg, about 0.3 mg/kg, about 1 mg/kg, about 2 mg/kg, about 3 mg/kg, about 5 mg/kg, or 10 mg/kg of individual body weight, or selected from a fixed dose of about 120 mg to about 480 mg, such as a fixed dose of 120 mg, 240 mg, 360 mg or 480 mg, preferably a fixed dose of 240 mg and 360 mg.

In one or more embodiments, in the method of the present invention, when administered, the cytotoxic anticancer drug is an anti-folate metabolism anticancer drug, wherein the single administration dose of the anti-folate metabolism anticancer drug is selected from about 200 mg/m² to about 800 mg/m², preferably 400 mg/m², 500 mg/m² or 600 mg/m².

In one or more embodiments, in the method of the present invention, when administered, the cytotoxic anticancer drug is a platinum anticancer drug, wherein the single administration dose of the platinum anticancer drug is selected from about 200 mg/m² to about 800 mg/m², preferably 400 mg/m², 500 mg/m² or 600 mg/m².

In one or more embodiments, in the method of the present invention, the dosing frequency of the anti-PD-1 antibody or the antigen binding fragment of the anti-PD-1 antibody is about once a week, once every two weeks, once every three weeks, once every four weeks, or once a month, preferably once every three weeks.

In one or more embodiments, in the method of the present invention, when administered, the dosing frequency of the anti-folate metabolism anticancer drug is about once a week, once every two weeks, once every three weeks, once every four weeks or once a month, preferably once every three weeks.

In one or more embodiments, in the method of the present invention, when administered, the dosing frequency of the platinum anticancer drug is about once a week, once every two weeks, once every three weeks, once every four weeks, or once a month, preferably once every three weeks.

In one or more embodiments, in the method of the present invention, the anti-PD-1 antibody or the antigen binding fragment of the anti-PD-1 antibody has an administration dose of a fixed dose of 240 mg or 360 mg and administered once every three weeks.

In one or more embodiments, in the method of the present invention, when administered, the anti-folate metabolism anticancer drug has an administration dose of 500 mg/m² and administered once every three weeks.

In one or more embodiments, in the method of the present invention, when administered, the platinum anticancer drug has an administration dose of 500 mg/m² and administered once every three weeks.

In one or more embodiments, in the method of the present invention, the dosing period of the anti-PD-1 antibody or the antigen binding fragment of the anti-PD-1 antibody, the anti-folate metabolism anticancer drug and/or the platinum anticancer drug is one week, two weeks, three weeks, one month, two months, three months, four months, five months, six months or longer, optionally, each dosing period can be the same or different, and the interval between each dosing period can be the same or different.

In one or more embodiments, in the method of the present invention, the anti-PD-1 antibody or the antigen binding fragment of the anti-PD-1 antibody, the anti-folate metabolism anticancer drug and the platinum anticancer drug are administered in a liquid dosage form, for example an injection, through parenteral route, for example, intravenous infusion.

In yet another aspect, the present invention provides a kit comprising one or more single-dose units of the anti-PD-1 antibody or the antigen binding fragment of the anti-PD-1 antibody and one or more single-dose units of the cytotoxic anticancer drug; preferably, the cytotoxic anticancer drug is an anti-folate metabolism anticancer drug and/or a platinum anticancer drug; preferably, the anti-PD-1 antibody or the antigen binding fragment of the anti-PD-1 antibody is according to any of the embodiments herein; preferably, the cytotoxic anticancer drug is according to any of the embodiments herein. More preferably, the anti-PD-1 antibody is toripalimab; the anti-folate metabolism anticancer drug is selected from methotrexate or pemetrexed, preferably pemetrexed; and the platinum anticancer drug is selected from cisplatin, carboplatin and oxaliplatin, preferably carboplatin.

In one or more embodiments, the kit comprises:

(I) one or more single-dose units of the anti-PD-1 antibody or the antigen binding fragment of the anti-PD-1 antibody, the single-dose unit comprises the anti-PD-1 antibody or the antigen binding fragment of the anti-PD-1 antibody at about 120 mg to about 480 mg, such as 120 mg, 240 mg, 360 mg or 480 mg, preferably 240 mg or 360 mg of; and

(II) one or more single-dose units of the anti-folate metabolism anticancer drug, the single-dose unit comprises the anti-folate metabolism anticancer drug at about 200 mg/m² to about 800 mg/m² of body surface area, for example 400 mg/m², 500 mg/m² or 600 mg/m² of body surface area, preferably 500 mg/m² of body surface area; and/or, one or more single-dose units of the platinum anticancer drug, the single-dose unit comprises the platinum anticancer drug at about 200 mg/m² to about 800 mg/m² of body surface area, for example 400 mg/m², 500 mg/m² or 600 mg/m² of body surface area, preferably 500 mg/m² of body surface area.

In one or more embodiments, the kit of the present invention comprises one or more single-dose units of the pharmaceutical composition according to any one of the embodiments of the present invention.

In one or more embodiments, the kit of the present invention further comprises instructions for instructing the method of use of the pharmaceutical composition.

### BRIEF DESCRIPTION OF THE DRAWINGS

FIG. 1: 1a: the largest change from baseline in tumor volume as assessed by RECIST v1.1 (n=38). The length of the bar represents the maximum reduction or minimum increase of the target lesion. #, the unproven partial response is classified as stable disease. *, patients with target lesions reduced by more than 30%, but with new lesions or non-target lesions being progressed. PD-L1 patients with IHC-positive tumor biopsy are marked with a “+” on the top. Patients with TMB≥10 muts/Mb are marked with a red triangle at the bottom. 1b: changes in individual tumor burden over time as assessed by RECIST v1.1 (n=38).

FIG. 2: clinical response as assessed by RECIST v1.1. 2a: the progression-free survival of 40 EGFR+NSCLC patients in this study; 2b: the overall survival of 40 EGFR+NSCLC patients in this study; 2c: the progression-free survival of patients with L585R mutation or exon 19 deletion; 2d: the overall survival of patients with L585R mutation or exon 19 deletion, the percentage of surviving patients is shown at the specified time point. Censored patients are marked with “|” in the figure. The number of patients in a dangerous state at the specified time point is shown below the x-axis. NE represents Not Estimated.

FIG. 3: the clinical response relationship related to tumor PD-L1 expression and tumor mutation burden. 3a: the threshold of PD-L1 status refers to tumor cells or immune cells with any intensity of membrane staining by JS311 IHC staining ≥1% or ≥10%, the tumor mutation burden (TMB) is calculated by summing the total cell mutations in the coding region after whole exome sequencing. 4.6 mutations/Mb is used as the cut-off value. 3b: the progression-free survival of PD-L1+ and PD-L1− patients/RECIST v1.1; 3c: the overall survival of PD-L1+ and PD-L1− patients; 3d: the progression-free survival of patients with TMB 4.6 Muts/Mb and TMB<4.6 Muts/Mb/RECIST v1.1; 3e: the overall survival of patients with TMB≥4.6 Muts/Mb and TMB<4.6 Muts/Mb. The PD-L1 positive state is defined as the presence of membrane staining of any intensity in ≥1% of tumor cells or immune cells by JS311 IHC staining. The percentage of surviving patients is shown at the specified time point. Censored patients are marked with “|” in the figure. The number of patients in a dangerous state at the specified time point is shown below the x-axis. NE represents Not Estimated.

FIG. 4: the genetic variation and frequency of 33 patients by whole exome sequencing (WES). Patients are grouped based on clinical response.

### DETAILED DESCRIPTION OF EMBODIMENTS

The present invention relates to a method for treating a malignant tumor. The method of the present invention comprises administering an anti-PD-1 antibody or an antigen binding fragment of the anti-PD-1 antibody to a patient in need. The malignant tumor of the present invention is non-small cell lung cancer. The present invention also relates to a method for using a biomarker to predict the efficacy of an anti-PD-1 antibody in a patient for treating a malignant tumor, especially non-small cell lung cancer.

### Terms

In order to make the present invention more readily understood, certain scientific and technological terms are specifically defined below. Unless otherwise explicitly stated elsewhere herein, the scientific and technological terms used herein have the meanings commonly understood by those of ordinary skill in the art to which the present invention belongs.

Unless the content is clearly stated otherwise, the singular forms “a”, “an” and “the” used in this specification and the appended claims include plural referents. Thus, for example, reference to “a polypeptide” includes a combination of two or more polypeptides.

“Administrate”, “dose” and “treat” refer to the introduction of a composition containing a therapeutic agent into a subject using any of various methods or delivery systems known to those skilled in the art. The administration route of an anti-PD-1 antibody includes intravenous, intramuscular, subcutaneous, peritoneal, spinal or other parenteral administration routes, such as injection or infusion. “Parenteral administration” refers to administration routes usually by injection other than enteral or local administration, including but not limited to intravenous, intramuscular, intraarterial, intrathecal, intralymphatic, intralesional, intracapsular, intraorbital, intracardiac, intradermal, intraperitoneal, transtracheal, subcutaneous, subepidermal, intraarticular, subcapsular, subarachnoid, intraspinal, intradural and intrasternal injection and infusion, and electroporation in vivo.

An “adverse effect” (AE) as described herein is any unfavorable and generally unintentional or undesirable sign, symptom, or disease associated with the use of medical treatment. For example, the adverse effect may be related to the activation of the immune system or the expansion of immune system cells in response to treatment. Medical treatments can have one or more related AEs, and each AE can have the same or different severity levels.

“Tumor burden” refers to the total amount of tumor material distributed throughout the body. Tumor burden refers to the total number of cancer cells or the total size of the tumors throughout the body. Tumor burden can be measured by a variety of methods known in the art, such as using a caliper after a tumor is removed from a subject, or using imaging techniques (such as ultrasound, bone scanning, computed tomography (CT) or magnetic resonance imaging (MRI) scan) to measure tumor size when the tumor is in the body.

The term “tumor size” refers to the total size of a tumor, which can be measured as the length and width of the tumor. The size of a tumor can be measured by a variety of methods known in the prior art, such as using a caliper after the tumor is removed from a subject, or using imaging techniques (such as bone scan, ultrasound, CT or MRI scan) to measure the size of the tumor when the tumor is in the body.

The terms “subject”, “individual”, and “object” include any organism, preferably animals, more preferably mammals (for example, rats, mice, dogs, cats, rabbits, etc.), and most preferably humans. The terms “subject” and “patient” are used interchangeably herein.

“Antibody” as described herein refers to any form of antibody that can achieve the desired biological activity or binding activity. Therefore, it is used in the broadest sense, but is not limited to monoclonal antibodies, polyclonal antibodies, multispecific antibodies, humanized full-length human antibodies, chimeric antibodies, and camel-derived single domain antibodies. An “antibody” specifically binds to an antigen and comprises at least two heavy (H) chains and two light (L) chains interconnected by disulfide bonds. Each heavy chain comprises a heavy chain variable region (VH) and a heavy chain constant region and the heavy chain constant region comprises three constant domains CH1, CH2 and CH3. Each light chain comprises a light chain variable region (VL) and a light chain constant region, and the light chain constant region comprises a constant domain CL. The VH and VL regions can be further subdivided into hypervariable regions called complementarity determining regions (CDRs), which are interspersed in more conservative regions called framework regions (FRs). In general, from N-terminus to C-terminus, both the light chain and heavy chain variable domains comprise FR1, CDR1, FR2, CDR2, FR3, CDR3, and FR4. Amino acids are usually assigned to each domain according to the definition in the following: Sequences of Proteins of Immunological Interest, Kabat et al.; National Institutes of Health, Bethesda. Md.; 5th edition; NIH publication number 91-3242 (1991): Kabat (1978) Adv. Prot. Chem. 32: 1-75; Kabat et al., (1977) J. Biol. Chem. 252: 6609-6616; Chothia et al., (1987) J Mol. Biol. 196: 901-917 or Chothia et al., (1989) Nature 341: 878-883.

The carboxy terminal part of the heavy chain can define the constant region that is mainly responsible for effector functions. Generally, human light chains are classified into a chains and λ chains. Human heavy chains are usually classified into μ, δ, γ, α, or ε, and the isotype of the antibody is defined as IgM, IgD, IgG, IgA, and IgE, respectively. The IgG subclass is well known to those skilled in the art and includes, but is not limited to, IgG1, IgG2, IgG, and IgG4.

The term “antibody” includes: naturally occurring and non-naturally occurring Ab; monoclonal and polyclonal Ab; chimeric and humanized Ab; human or non-human Ab; fully synthetic Ab; and single-stranded Ab. Non-human Ab can be humanized by recombinant methods to reduce their immunogenicity in human.

Unless expressly stated otherwise, the “antibody fragment” or “antigen binding fragment” as described herein refers to an antigen binding fragment of an antibody, that is, an antibody fragment that retains the ability of a full-length antibody to specifically bind to an antigen, such as a fragment that retains one or more CDR regions. Examples of antigen binding fragments include but are not limited to Fab, Fab′, F(ab′)2 and Fv fragment; double-stranded antibody; linear antibody; single-stranded antibody molecule; nanobody and multispecific antibody formed from antibody fragment.

“Chimeric antibody” refers to the following antibodies and fragments of the following antibodies: in which a part of the heavy chain and/or light chain is the same as or homologous to the corresponding sequence in an antibody derived from a specific species (such as human) or belonging to a specific antibody class or subclass, while the rest of the chain is the same as or homologous to the corresponding sequence in an antibody derived from another species (such as mouse) or belonging to another antibody class or subclass, as long as the chimeric antibody exhibits the desired biological activity.

“Human antibody” refers to an antibody that comprises only human immunoglobulin sequences. If the human antibody is produced in a mouse, mouse cell, or hybridoma derived from a mouse cell, it may comprises murine carbohydrate chains. Similarly, “mouse antibody” or “rat antibody” refers to an antibody that only comprises mouse or rat immunoglobulin sequences, respectively.

“Humanized antibody” refers to a form of antibody that comprises sequences derived from a non-human (such as murine) antibody and a human antibody. Such antibody comprises the minimal sequence derived from a non-human immunoglobulin. Generally, a humanized antibody would comprise substantially all of at least one and usually two variable domains, wherein all or substantially all of the hypervariable loops correspond to the hypervariable loops of non-human immunoglobulins, and all or substantially all of the FR regions are the FR regions of human immunoglobulins. A humanized antibody optionally also comprises at least a portion of an immunoglobulin constant region (Fc) (usually a human immunoglobulin constant region).

As used herein, the term “cancer” or “malignant tumor” refers to a wide range of diseases characterized by the uncontrolled growth of abnormal cells in the body. Unregulated cell division, growth division and growth lead to the formation of malignant tumors, which invade adjacent tissues and can also metastasize to remote parts of the body through the lymphatic system or bloodstream. Examples of cancers that are suitable for treatment or prevention using the method, drug, and kit of the present invention include, but are not limited to, carcinoma, lymphoma, leukemia, blastoma, and sarcoma. More specific examples of cancers include squamous cell carcinoma, myeloma, small cell lung cancer, non-small cell lung cancer, glioma, Hodgkin's lymphoma, non-Hodgkin's lymphoma, acute myelogenous leukemia, multiple myeloma, gastrointestinal (tract) cancer, kidney cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, nasopharyngeal cancer, cervical cancer, brain cancer, gastric cancer, bladder cancer, hepatoma, breast cancer, colon cancer and head and neck cancer.

Herein, the term “tumor mutation burden (TMB)” refers to the total number of somatic gene coding errors, base substitutions, gene insertion or deletion errors detected per million bases. In some embodiments of the present invention, tumor mutation burden (TMB) is estimated by analyzing somatic mutations (including coding base substitutions and megabase insertions of the studied panel sequence).

The term “non-small cell lung cancer” divides non-small cell lung cancer (NSCLC) into three categories based on the appearance and other characteristics of cancer cells: squamous cell carcinoma (SCC), adenocarcinoma and large cell carcinoma (LCC). SCC accounts for approximately 25%-30% of all lung cancer cases. SCC is closely related to smoking and usually occurs in the central area of the lung; adenocarcinoma accounts for about 40% of all lung cancer cases, and this type of cancer usually occurs in the outer area of the lung; LCC accounts for about 10%-15% of all lung cancer cases, and LCC patients usually have rapid tumor growth and poor prognosis. Other uncommon types of lung cancer include carcinoid tumors, adenoid cystic carcinoma, hamartoma, lymphoma, and sarcoma.

The term “immunotherapy” refers to the treatment of a subject who has a disease or is at risk of infection or suffering from recurrence of a disease by methods that include inducing, enhancing, suppressing, or otherwise modifying the immune response. “Treatment” or “therapy” of a subject refers to any type of intervention or process performed on a subject, or administration of an active agent to a subject, with the purpose of reversing, alleviating, ameliorating, slowing or preventing the onset, progression, severity or recurrence of the symptoms, complications or conditions, or biochemical indicators related to diseases.

“Programmed death receptor-1 (PD-1)” refers to an immunosuppressive receptor belonging to the CD28 family. PD-1 is mainly expressed on previously activated T cells in the body and binds to two ligands, PD-L1 and PD-L2. The term “PD-1” as used herein includes human PD-1 (hPD-1) and variants, isotypes and species homologs of hPD-1, and analogs that have at least one epitope in common with hPD-1.

A “therapeutically effective amount” or “therapeutically effective dose” of a drug or therapeutic agent is any amount of a drug that protects a subject from the onset of a disease or promotes the regression of a disease when used alone or in combination with another therapeutic agent. The regression of the disease is evidenced by the reduction in the severity of the symptoms of the disease, the increase in the frequency and duration of the asymptomatic period of the disease, or the prevention of injury or disability caused by the pain of the disease. The ability of a therapeutic agent to promote disease regression can be evaluated using a variety of methods known to those skilled in the art, such as determining the activity of the agent in human subjects during clinical trials, in animal model systems that predict the efficacy in human, or by in vitro assays.

A therapeutically effective amount of a drug includes a “prophylactically effective amount”, that is, any amount of a drug that inhibits the development or recurrence of a cancer when administered to a subject at risk of developing the cancer or a subject suffering from recurrence of the cancer, alone or in combination with an antitumor agent.

“Biotherapeutic agent” refers to biomolecules, such as antibodies or fusion proteins, that block ligand/receptor signaling in any biological pathway that supports tumor maintenance and/or growth or inhibits antitumor immune responses.

Unless expressly stated otherwise, “CDR” as used herein means that the immunoglobulin variable region is a complementarity determining region defined using the Kabat numbering system.

“Therapeutic anti-PD-1 monoclonal antibody” refers to an antibody that specifically binds to the mature form of specific PD-1 expressed on the surface of certain mammalian cells. Mature PD-1 has no pre-secretory leader sequence, or leader peptide. The terms “PD-1” and “mature PD-1” are used interchangeably herein, and unless clearly defined otherwise, or clearly seen from the context, they should be understood as the same molecule.

As described herein, a therapeutic anti-human PD-1 antibody or anti-hPD-1 antibody refers to a monoclonal antibody that specifically binds to mature human PD-1.

“Framework region” or “FR” as described herein refers to a immunoglobulin variable region that does not include CDR regions.

“Isolated antibody or antigen binding fragment of the isolated antibody” refers to a purified state and in this case a designated molecule does not substantially comprise other biological molecules, such as nucleic acids, proteins, lipids, carbohydrates or other materials (such as cell debris or growth culture medium).

“Patient”, “sufferer” or “subject” refers to any single subject in need of medical treatment or participating in clinical trials, epidemiological studies or used as a control, usually mammals, including humans and other mammals, such as horses, cows, dogs or cats.

The “RECIST 1.1 efficacy criteria” as described herein refers to the definition of Eisenhauver et al., E. A. et al., Eur. J Cancer 45: 228-247 (2009) for target lesion or non-target lesion based on the background of the measured response. Before immunotherapy, it was the most commonly used criteria for evaluating the efficacy for solid tumors. However, with the advent of the age of immunization, there have been many problems that have not appeared in tumor evaluation before. Therefore, based on the emerging phenomenon caused by immunotherapy itself, in 2016, the RECIST working group reviewed the existing “RECIST v.1.1”. After revision, a new judgment criteria, the “irRECIST criteria” described herein, was proposed, which aimed to better evaluate the efficacy of immunotherapy drugs.

The term “ECOG” scoring criteria is an indicator of physical strength of a patient to understand the general health and tolerance to treatment of the patient. The scores for ECOG physical strength status scoring criteria are: 0 points, 1 point, 2 points, 3 points, 4 points and 5 points. A score of 0 refers that the activity ability is completely normal, and there is no difference comparing to the activity ability before the onset of a disease. A score of 1 refers to the ability to move around freely and engage in light physical activities, including general housework or office work, but not to engage in heavy physical activities.

“Sustained response” refers to the sustained therapeutic effect after cessation of the therapeutic agent or combination therapy described herein. In some embodiments, the sustained response has a duration that is at least the same as the duration of the treatment or at least 1.5, 2.0, 2.5, or 3 times the duration of the treatment.

“Tissue section” refers to a single part or piece of a tissue sample, such as a tissue slice cut from a sample of normal tissue or tumor.

“Treatment” of a cancer as described herein refers that the treatment regimen described herein (for example, administration of an anti-PD-1 antibody) is administrated to a subject suffering from the cancer or having been diagnosed with the cancer to achieve at least one positive therapeutic effect (for example, decrease in cancer cell number, decrease in tumor volume, decrease in the rate of cancer cell infiltration into surrounding organs, or decrease in the rate of tumor metastasis or tumor growth). The positive treatment effect in cancer can be measured in a variety of ways (see W. A. Weber, J. Nucl. Med., 50: 1S-10S (2009)). For example, regarding tumor growth inhibition, according to the NCI criteria, T/C≤42% is the minimum level of antitumor activity. T/C (%)=median of the volume of treated tumor/median of the volume of control tumor×100. In some embodiments, the therapeutic effect achieved by the combination of the present invention is any one of PR, CR, OR, PFS, DFS, and OS. PFS (also called “time to tumor progression”) refers to the length of time during and after treatment that a cancer does not grow, and includes the length of time during which a patient experiences CR or PR and the length of time during which a patient experiences SD. DFS refers to the length of time during and after treatment that a patient remains disease-free. OS refers to the increase in life expectancy compared to the initial or untreated individual or patient. In some embodiments, the response to the combination of the present invention is any of PR, CR, PFS, DFS, OR, or OS, which is assessed using the RECIST 1.1 efficacy criteria. The treatment regimen of the combination of the present invention that is effective in treating cancer patients may vary according to various factors, such as the patient's disease state, age, weight, and the ability of the therapy to stimulate the subject's anticancer response. Although the embodiment of the present invention may not achieve an effective positive therapeutic effect in every subject, it should be effective and achieve a positive therapeutic effect in a statistically significant number of subjects.

The terms “dosing route” and “dosing regimen” are used interchangeably, and refer to the dosage and time of use of each therapeutic agent in the combination of the present invention.

The term “immunohistochemistry (IHC)” refers to a method which use the principle of specific binding of antigens and antibodies to determine the intracellular antigens (polypeptides and proteins) of tissues by chemical reactions that make the chromogenic reagents (fluorescein, enzymes, metal ions, isotopes) labeled with antibodies develop, and determine the location, identity and relative amount of the intracellular antigens. In some embodiments of the present invention, before using the anti-PD-1 antibody treatment, the tumor tissue samples from a subject are detected for PD-L1 using anti-human PD-L1 antibody SP142 (Roche, Cat No: M4422) in a staining experiment. In some embodiments, tumor cell membrane staining intensity ≥1% is defined as PD-L1 positive.

In the following paragraphs, various aspects of the present invention are described in further detail.

### Anti-PD-1 Antibody

Here, “PD-1 antibody” refers to any chemical compound or biological molecule which binds to PD-1 receptor, blocks the binding of PD-L1 expressed on cancer cells with PD-1 expressed on immune cells (T, B, NK cells) and preferably can also block the binding of PD-L2 expressed on cancer cells with PD-1 expressed on immune cells. Alternative terms or synonyms for PD-1 and its ligands include: for PD-1, there are PDCD1, PD1, CD279 and SLEB2; for PD-L1, there are PDCD1L1, PDL1, B7-H1, B7H1, B7-4, CD274 and B7-H; and for PD-L2, there are PDCD1L2, PDL2, B7-DC and CD273. In any of the treatment method, drug and use of the present invention for treating human individuals, the PD-1 antibody blocks the binding of human PD-L1 with human PD-1, and preferably blocks the binding of both human PD-L1 and PD-L2 with human PD1. The amino acid sequence of human PD-1 can be found in the NCBI locus numbering: NP_005009. The amino acid sequences of human PD-L1 and PD-L2 can be found in the NCBI locus numbering: NP_054862 and NP_079515, respectively.

Herein, when referring to “anti-PD-1 antibody”, unless stated or described otherwise, the term includes antigen binding fragments of the anti-PD-1 antibody.

The anti-PD-1 antibody suitable for any use, therapy, drug and kit of the present invention binds to PD-1 with high specificity and high affinity, blocks the binding of PD-L1/2 with PD-1, and inhibits PD-1 signal transduction, so as to achieve an immunosuppressive effect. In any of the use, therapy, drug, and kit disclosed herein, the anti-PD-1 antibody includes the full-length antibody itself, as well as a antigen binding portion or fragment which binds to PD-1 receptor and exhibits functional properties similar to intact Abs in terms of inhibiting ligand binding and upregulating the immune system. In some embodiments, the anti-PD-1 antibody or the antigen binding fragment of the anti-PD-1 antibody is an anti-PD-1 antibody or an antigen binding fragment of the anti-PD-1 antibody that cross-competes with toripalimab for binding to human PD-1. In other embodiments, the anti-PD-1 antibody or the antigen binding fragment of the anti-PD-1 antibody is a chimeric, humanized or human Ab or an antigen binding fragment of the anti-PD-1 antibody. In certain embodiments for treating a human individual, the Ab is a humanized Ab.

In some embodiments, the anti-PD-1 antibody used in any of the use, therapy, drug, and kit described in the present invention include a monoclonal antibody (mAb) or an antigen binding fragment of the monoclonal antibody, which specifically binds to PD-1, preferably specifically binds to human PD-1. The mAb can be a human antibody, a humanized antibody, or a chimeric antibody, and can comprise a human constant region. In some embodiments, the constant region is selected from the group consisting of human IgG1, IgG2, IgG3, and IgG4 constant regions; preferably, the anti-PD-1 antibody or the antigen binding fragment of the anti-PD-1 antibody suitable for any of the use, therapy, drug and kit of the present invention comprises a heavy chain constant region of human IgG1 or IgG4 isotype, more preferably a human IgG4 constant region. In some embodiments, the sequence of the IgG4 heavy chain constant region of the anti-PD-1 antibody or the antigen binding fragment of the anti-PD-1 antibody comprises S228P mutation, which replaces the serine residue in hinge region with the proline residue which is usually present at the corresponding position of an IgG1 isotype antibody.

Preferably, in any embodiment of the use, therapy, drug and kit of the present invention, the PD-1 antibody is a monoclonal antibody or an antigen binding fragment of the monoclonal antibody, and has light chain CDRs with the amino acids shown in SEQ ID NOs: 1, 2 and 3, and heavy chain CDRs with the amino acids shown in SEQ ID NOs: 4, 5, and 6.

More preferably, in any embodiment of the use, therapy, drug and kit of the present invention, the PD-1 antibody specifically binds to human PD-1 and comprises: a monoclonal antibody comprising (a) a light chain variable region of SEQ ID NO: 7 and (b) a heavy chain variable region of SEQ ID NO: 8.

Further preferably, in any embodiment of the use, therapy, drug and kit of the present invention, the PD-1 antibody specifically binds to human PD-1 and comprises: a monoclonal antibody comprising (a) a light chain of SEQ ID NO: 9 and (b) a heavy chain of SEQ ID NO: 10.

Table A below provides the amino acid sequence numeration of the light chain CDRs and heavy chain CDRs of an exemplary anti-PD-1 antibody mAb used in the use, therapy, drug, and kit of the present invention:

TABLE A
Light and heavy chain CDRs of an exemplary
anti-human PD-1 antibody

| | |
|---|---|
| LCDR1 | SEQ ID NO: 1 |
| LCDR2 | SEQ ID NO: 2 |
| LCDR3 | SEQ ID NO: 3 |
| HCDR1 | SEQ ID NO: 4 |
| HCDR2 | SEQ ID NO: 5 |
| HCDR3 | SEQ ID NO: 6 |

Examples of the anti-PD-1 antibody which binds to human PD-1 and can be used in the use, therapy, drug, and kit of the present invention are described in WO 2014206107. The human PD-1 mAb which can be used as an anti-PD-1 antibody in the use, therapy, drug and kit of the present invention comprises any of the anti-PD-1 antibodies described in WO 2014206107, comprising: toripalimab, which has the structure described in the WHO Drug Information (Vol. 32, No. 2, pages 372-373 (2018)) and comprises humanized IgG4 mAb having a light chain and heavy chain with the amino acid sequence shown in SEQ ID NOs: 9 and 10, respectively. In a preferred embodiment, the anti-PD-1 antibody which can be used in any of the use, therapy, drug, and kit described in the present invention is selected from the humanized antibodies 38, 39, 41, and 48 described in WO 2014206107. In a particularly preferred embodiment, the anti-PD-1 antibody which can be used in any of the use, therapy, drug, and kit described in the present invention is toripalimab.

The anti-PD-1 antibodies which can be used in any of the use, therapy, drug and kit described in the present invention also comprise nivolumab and pembrolizumab which have been approved by the FDA.

In certain embodiments, the anti-PD-1 antibody which can be used in any of the use, therapy, drug and kit described in the present invention also comprises anti-PD-L1 monoclonal antibodies (such as nivolumab, pembrolizumab, toripalimab, sintilimab, camrelizumab, tislelizumab and cemiplimab) which specifically bind to PD-L1 to block the binding of PD-L1 with PD-1.

“PD-L1” expression or “PD-L2” expression as described herein refers to any detectable expression level of a specific PD-L protein on the surface of a cell or a specific PD-L mRNA in a cell or tissue. PD-L protein expression can be detected by diagnostic PD-L antibody in IHC analysis of tumor tissue sections or by flow cytometry. Alternatively, the PD-L protein expression of tumor cells can be detected by PET imaging using a binding agent that specifically binds to the desired PD-L target (such as PD-L1 or PD-L2).

For methods for quantifying PD-L1 protein expression in IHC analysis of tumor tissue sections, see the following but not limited to Thompson, R. H. et al., PNAS 101 (49): 17174-17179 (2004); Taube, J. M. et al., Sci Transi Med 4, 127ra37 (2012); and Toplian, S. L. et al., New Eng. J. Med. 366 (26): 2443-2454 (2012) and so on.

One method uses a simple binary endpoint where PD-L1 expression is positive or negative, wherein a positive result is defined by the percentage of tumor cells showing histological evidence of cell surface membrane staining. Counting tumor tissue sections as at least 1% of the total tumor cells is defined as positive for PD-L1 expression.

In another method, PD-L1 expression in tumor tissue sections is quantified in tumor cells and in infiltrating immune cells. The percentages of tumor cells and infiltrating immune cells exhibiting membrane staining are separately quantified as <1% 1% to 50%, and subsequent 50% up to 100%. For tumor cells, if the score is <1%, the PD-L1 expression is negative, and if the score is ≥1%, it is positive.

In some embodiments, the expression level of PD-L1 by malignant cells and/or by infiltrating immune cells within the tumor is determined to be “overexpression” or “elevated” based on a comparison with the expression level of PD-L1 by an appropriate control. For example, the protein or mRNA expression level of the control PD-L1 can be a quantitative level in a non-malignant cell of the same type or in a section from a matched normal tissue.

### Cytotoxic Anticancer Drugs

In some embodiments of the present invention for the treatment of non-small cell lung cancer, cytotoxic anticancer drugs comprise drugs that damage the structure and function of DNA, drugs that affect nucleic acid biosynthesis, drugs that interfere with the transcription process and inhibit RNA synthesis, and drugs that act on DNA replication topoisomerase inhibitors and that affect protein synthesis and function. Among them, drugs that damage the structure and function of DNA comprise nitrogen mustard, cyclophosphamide, and platinum anticancer drugs. Drugs that affect nucleic acid biosynthesis comprise thymidylate synthetase inhibitors, DNA polymerase inhibitors, anti-folate metabolism anticancer drugs, nucleotide reductase inhibitors, and purine nucleotide synthetase inhibitors. In some embodiments, the cytotoxic anticancer drugs of the present invention are selected from anti-folate metabolism anticancer drugs and platinum anticancer drugs.

### Anti-Folate Metabolism Anticancer Drugs

In some embodiments of the present invention for the treatment of non-small cell lung cancer, the anti-folate metabolism anticancer drug is selected from methotrexate and pemetrexed, preferably pemetrexed. Pemetrexed is an anti-folate formulation with a pyrrolopyrimidine group as the core in the structure. Pemetrexed inhibits cell replication by disrupting the normal intracellular metabolic process dependent on folate, thereby inhibiting tumor growth. Pemetrexed is a compound having the structure represented by formula (I).

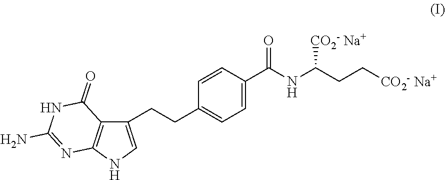

In some embodiments of the present invention, pemetrexed can also refer to a composition comprising a therapeutically effective amount of a compound represented by formula (I), a free base of the compound, or a pharmaceutically acceptable salt of the compound, and a pharmaceutically acceptable excipient.

### Platinum Anticancer Drugs

In some embodiments of the present invention for the treatment of non-small cell lung cancer, the platinum anticancer drug is selected from cisplatin, carboplatin and oxaliplatin; preferably, carboplatin. Carboplatin was discovered by Clear et al. in 1980 and was first marketed in the UK in 1986 and approved by the US FDA in 1989. The application of carboplatin was gradually promoted. China approved the production of carboplatin powder and injection in 1990. Carboplatin is a second-generation platinum compound. The biochemical characteristics of carboplatin are similar to cisplatin. Carboplatin is a new drug that has received extensive attention in recent years and is a cell cycle non-specific drug. Carboplatin mainly acts on the N7 and 06 atoms of the guanine of DNA, causing cross-linking between and within the DNA chain, destroying DNA molecules, preventing DNA spiral melting, interfering with DNA synthesis, thereby causing cytotoxicity. Carboplatin is a compound having the structure represented by formula (II).

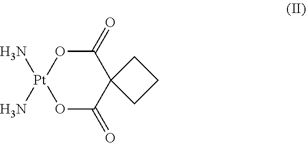

In some embodiments of the present invention, carboplatin can also refer to a composition comprising a therapeutically effective amount of a compound represented by formula (II) or a pharmaceutically acceptable salt of the compound, and a pharmaceutically acceptable excipient.

### Pharmaceutical Compositions

The present invention provides a pharmaceutical composition comprising the anti-PD-1 antibody, the anti-folate metabolism anticancer drug, the platinum anticancer drug, and other pharmaceutically acceptable carriers as described herein. In some embodiments, the present invention also provides a pharmaceutical composition comprising the anti-PD-1 antibody, the anti-folate metabolism anticancer drug and other pharmaceutically acceptable carriers as described herein.

In some embodiments, the anti-PD-1 antibody of the present invention may be according to any of the embodiments herein, more preferably, the anti-PD-1 antibody of the present invention is an antibody comprising light chain CDRs with the amino acids shown in SEQ ID NOs: 1, 2 and 3 and heavy chain CDRs with the amino acids shown in SEQ ID NO: 4, 5 and 6; more preferably, the anti-PD-1 antibody of the present invention is a monoclonal antibody comprising a light chain variable region shown in SEQ ID NO: 7 and a heavy chain variable region shown in SEQ ID NO: 8; more preferably, the anti-PD-1 antibody of the present invention is a monoclonal antibody comprising a light chain shown in SEQ ID NO: 9 and a heavy chain shown in SEQ ID NO: 10; more preferably, the anti-PD-1 antibody of the present invention is the humanized antibodies 38, 39, 41 and 48 described in WO 2014206107; most preferably, the anti-PD-1 antibody of the present invention is toripalimab. The anti-folate metabolism anticancer drug may be according to any of the embodiments herein, more preferably, methotrexate or pemetrexed; preferably, pemetrexed. The platinum anticancer drug is selected from cisplatin, carboplatin and oxaliplatin; preferably, carboplatin.

As described in the present invention, “pharmaceutically acceptable carrier” comprises any and all solvents, dispersion media, coatings, antibacterial and antifungal agents, isotonic and absorption delaying agents and the like that are physiologically compatible. Preferably, the carrier suitable for the composition comprising the anti-PD-1 antibody is suitable for intravenous, intramuscular, subcutaneous, parenteral, spinal or epidermal administration, such as by injection or infusion, and the carrier used in the composition comprising other anticancer agents is suitable for parenteral administration, such as oral administration. The pharmaceutical composition of the present invention may comprises one or more pharmaceutically acceptable salts, antioxidants, water, non-aqueous carriers, and/or adjuvants such as preservatives, wetting agents, emulsifying agents and dispersing agents.

The content of anticancer active ingredients (for example, the anti-PD-1 antibody, the anti-folate metabolism anticancer drug and the platinum anticancer drug described herein) in each dose of the pharmaceutical composition of the present invention is usually the amount of each of these anticancer active ingredients in a single administration. For example, for each fixed dose of 240 mg of the anti-PD-1 antibody described herein, each dose of the pharmaceutical composition may contain 240 mg of the anti-PD-1 antibody. Of course, for oral tablets, for example, the 240 mg of anti-PD-1 antibody can be divided into 2 or more tablets, as long as all these tablets are taken at the time of administration to achieve a dosage of 240 mg.

### Dosage and Dosing Regimen

The choice of dosing regimen (also referred to herein as an administration regimen) for the drug combination of the present invention depends on several factors, including the entity serum or tissue turnover rate, symptom level, overall immunogenicity, and accessibility of target cells, tissues or organs of the individual being treated. Preferably, the dosing regimen maximizes the amount of each therapeutic agent delivered to the patient with an acceptable level of side effects. Therefore, the dosage and dosing frequency of each biotherapeutic agent and chemotherapeutic agent depends in part on the specific therapeutic agent, the severity of the cancer being treated, and the characteristics of the patient. Guidance on choosing the appropriate dosage of antibodies, cytokines, and small molecules can be obtained. See, for example, Wawrzynczak (1996) Antibody Therapy. Bios Scientific Pub. Ltd. Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348: 601-608; Milgrom et al. (1999) New Engl. J. Med. 341: 1966-1973; Slamon et al. (2001) New Engl. J. Med. 344: 783-792; Beniaminovitz et al. (2000) New Engl. J. Med. 342: 613-619; Ghosh et al. (2003) New Engl. J. Med. 348: 24-32; Lipsky et al. (2000) New Engl. J. Med. 343: 1594-1602; Physicians' Desk Reference 2003 (Physicians' Desk Reference, 57th Ed); Medical Economics Company; ISBN: 1563634457; 57th edition (November 2002). The determination of the appropriate dosage regimen can be performed by the clinician, for example, with reference to the parameters or factors known or suspected to affect the treatment or expected to affect the treatment in the art, and this will depend on, for example, the patient's clinical history (e.g., previous treatment), the type and stage of the cancer being treated, and biomarkers that respond to one or more therapeutic agents in the combination therapy.

Each therapeutic agent of the pharmaceutical combination of the present invention can be administered simultaneously (i.e, in the same pharmaceutical composition), concurrently (i.e, in separate pharmaceutical formulations, administered one after the other in any order), or sequentially in any order. When the therapeutic agents in the drug combination can be administered in different dosage forms (one drug is a tablet or capsule and the other drug is a sterile liquid formulation) and/or in a different dosing schedule (for example, the chemotherapeutic agent is administered at least daily and the biotherapeutic agent is administered less frequently (for example, once a week, once every two weeks, or once every three weeks)), sequential administration is particularly useful.

In some embodiments, at least one therapeutic agent in the drug combination is administered using the same dosage regimen (therapeutic dosage, frequency, and duration) that is commonly used when the agent is used to treat the same tumor as a single treatment. In other embodiments, the patient receives a smaller total amount of at least one therapeutic agent in the combination therapy than in the monotherapy of the at least one therapeutic agent, such as a smaller dosage, a less frequent dosage, and/or a shorter duration of treatment.

Each therapeutic agent in the pharmaceutical combination of the present invention can be administered orally or parenterally, including administered via intravenous, intramuscular, intraperitoneal, subcutaneous, rectal, topical, and transdermal route.

The anti-PD-1 antibody of the present invention can be administered by continuous infusion or by interval doses. The single administration dose can range from about 0.01 to about 20 mg/kg of individual body weight, about 0.1 to about 10 mg/kg of individual body weight, or a fixed dose of about 120 mg to about 480 mg. For example, the dose can be about 0.1, about 0.3, about 1, about 2, about 3, about 4, about 5, about 6, about 7, about 8, about 9, or about 10 mg/kg of individual body weight, or a fixed dose of about 120 mg, 240 mg, 360 mg or 480 mg. The dosing regimen is usually designed to achieve a exposure which results in sustained receptor occupancy (RO) based on the typical pharmacokinetic properties of Ab. A representative dosing regimen may be about once a week, about once every two weeks, about once every three weeks, about once every four weeks, about once a month or longer. In some embodiments, the anti-PD-1 antibody is administered to the individual about once every three weeks.

In some embodiments, the anti-PD-1 antibody of the present invention is toripalimab, and its single administration dose is selected from about 1 to about 5 mg/kg of individual body weight. In some embodiments, the single administration dose of toripalimab is selected from about 1 mg/kg, 2 mg/kg, 3 mg/kg, 3 mg/kg, 4 mg/kg, and 5 mg/kg of individual body weight, or a fixed dose of 120 mg, 240 mg and 360 mg, and is administered intravenously. In some preferred embodiments, toripalimab is administered as a liquid drug, and the selected dose of the drug is administered by intravenous infusion over a period of 30 to 60 minutes. In some embodiments, toripalimab is administered at about 3 mg/kg or a fixed dose of about 240 mg, once every three weeks (Q3W), by intravenous infusion over a period of 30 minutes. In some embodiments, toripalimab is administered at about 4.5 mg/kg or a fixed dose of about 360 mg, once every three weeks (Q3W), by intravenous infusion over a period of 30 minutes.

The anti-folate metabolism anticancer drug of the present invention is administered at its approved or recommended dose, and treatment is continued until a clinical effect is observed or until unacceptable toxicity or progressive disease occurs. In some embodiments, the anti-folate metabolism anticancer drug of the present invention is pemetrexed, and its single administration dose is selected from about 200 mg to about 800 mg/m² of body surface area. In some embodiments, the single administration dose of pemetrexed is selected from any dose of about 300 mg/m², 400 mg/m², 500 mg/m², 600 mg/m² and 700 mg/m² of body surface area. A representative dosing regimen may be about once a week, once every two weeks, once every three weeks, once every four weeks, or once a month. In some embodiments, pemetrexed is administered to the individual once every three weeks. In some embodiments, pemetrexed is administered at about 500 mg/m² of body surface area, once every three weeks (Q3W).

The platinum anticancer drug of the present invention is administered at its approved or recommended dose, and the treatment is continued until the disease maintenance stage is entered, or until unacceptable toxicity or progressive disease occurs. In some embodiments, the platinum anticancer drug of the present invention is carboplatin, and its single administration dose is selected from about 200 mg to about 800 mg/m² of body surface area. In some embodiments, the single administration dose of carboplatin is selected from any dose of about 300 mg/m², 400 mg/m², 500 mg/m², 600 mg/m² and 700 mg/m² of body surface area. A representative dosing regimen may be about once a week, once every two weeks, once every three weeks, once every four weeks, or once a month. In some embodiments, carboplatin is administered to the individual once every three weeks. In some embodiments, carboplatin is administered at about 500 mg/m² of body surface area, once every three weeks.

In some embodiments, toripalimab is administered at a fixed dose of about 240 mg, Q3W, pemetrexed is administered at about 500 mg/m² of body surface area, Q3W, and carboplatin is administered at about 500 mg/m² of body surface area, Q3W. In some embodiments, toripalimab is administered at a fixed dose of about 360 mg, Q3W, pemetrexed is administered at about 500 mg/m² of body surface area, Q3W, and carboplatin is administered at about 500 mg/m² of body surface area, Q3W. In some embodiments, toripalimab is administered at a fixed dose of about 240 mg, Q3W, and pemetrexed is administered at a fixed dose of about 200 mg, Q3W. In some embodiments, toripalimab is administered at a fixed dose of about 360 mg, Q3W, and pemetrexed is administered at a fixed dose of about 200 mg. Q3W.

In some embodiments, on the day of toripalimab administration, pemetrexed may be administered before or after toripalimab administration, and carboplatin may be administered before or after toripalimab administration.

The dosing period of the anti-PD-1 antibody and the cytotoxic anticancer drug of the present invention can be the same or different, which is one week, two weeks, three weeks, one month, two months, three months, four months, five months, half a year or longer, optionally, each dosing period can be the same or different, and the interval between each dosing period can be the same or different. For example, in some embodiments, toripalimab is administered at a fixed dose of about 240 mg once every three weeks, and pemetrexed is administered at about 500 mg/m² of body surface area every three weeks, and carboplatin is administered at about 500 mg/m² of body surface area every three weeks. The three dosing periods are all three weeks.

### Treatment Methods and Uses

The present invention relates to the use of an anti-PD-1 antibody or an antigen binding fragment of the anti-PD-1 antibody and a cytotoxic anticancer drug in the preparation of a medicine for treating non-small cell lung cancer.

The present invention also comprises a method for preventing or treating non-small cell lung cancer, wherein the method comprises the combined administration of an anti-PD-1 antibody or an antigen binding fragment of the anti-PD-1 antibody and a cytotoxic anticancer drug or the pharmaceutical composition according to the present invention to an individual in need.

The non-small cell lung cancer may be according to any of the foregoing embodiments; further, the non-small cell lung cancer is a non-small cell lung cancer that has failed EGFR-TKI treatment. Still further, the non-small cell lung cancer is advanced or recurrent non-small cell lung cancer that has failed EGFR-TKI treatment and has EGFR mutations.

Preferably, the method, use and pharmaceutical composition described in any embodiment of the present invention are particularly suitable for non-small cell lung cancer with positive PD-L1 expression.

Preferably, the method, use and pharmaceutical composition described in any embodiment of the present invention are particularly suitable for non-small cell lung cancer with deletion of EGFR exon 19 or L858R mutation in exon 21; preferably, non-small cell lung cancer with L858R mutation in EGFR exon 21.

The preferred anti-PD-1 antibody for non-small cell lung cancer may be according to any of the embodiments herein, more preferably, the anti-PD-1 antibody of the present invention is an antibody comprising light chain CDRs with the amino acids shown in SEQ ID NOs: 1, 2 and 3 and heavy chain CDRs with the amino acids shown in SEQ ID NO: 4, 5 and 6; more preferably, the anti-PD-1 antibody of the present invention is a monoclonal antibody comprising a light chain variable region shown in SEQ ID NO: 7 and a heavy chain variable region shown in SEQ ID NO: 8; more preferably, the anti-PD-1 antibody of the present invention is a monoclonal antibody comprising a light chain shown in SEQ ID NO: 9 and a heavy chain shown in SEQ ID NO: 10; more preferably, the anti-PD-1 antibody of the present invention is the humanized antibodies 38, 39, 41 and 48 described in WO 2014206107; most preferably, the anti-PD-1 antibody of the present invention is toripalimab.

The preferred cytotoxic anticancer drug may be according to any of the embodiments herein, preferably anti-folate metabolism anticancer drugs and platinum anticancer drugs. The anti-folate metabolism anticancer drug may be according to any of the embodiments herein, more preferably, methotrexate or pemetrexed; preferably, pemetrexed, the platinum anticancer drug is selected from cisplatin, carboplatin and oxaliplatin; preferably, carboplatin.

In a particularly preferred embodiment, the present invention provides a method for treating non-small cell lung cancer, the method comprises administering a therapeutically effective amount of toripalimab, pemetrexed and carboplatin to a patient with non-small cell lung cancer; preferably, the patient is positive for PD-L1 expression. In certain embodiments, preferably, the patient with non-small cell lung cancer has failed EGFR-TKI treatment, EGFR exon 19 deletion or L858R mutation in exon 21, preferably L858R mutation in EGFR exon 21. Preferably, the dosing regimen of the treatment method is according to any of the embodiments herein.

### Kits

The present invention also provides a kit comprising one or more single-dose units of the anti-PD-1 antibody or the antigen binding fragment of the anti-PD-1 antibody according to any of the embodiments herein and one or more single-dose units of the cytotoxic anticancer drug according to any of the embodiments herein. Preferably, the cytotoxic anticancer drug is the anti-folate metabolism anticancer drug and/or platinum anticancer drug according to any of the embodiments herein.

In some embodiments, the kit of the present invention comprises toripalimab, pemetrexed and carboplatin as the anticancer active agents in the kit. In some other embodiments, the kit of the present invention comprises toripalimab and pemetrexed as the anticancer active agents in the kit.

The anticancer active ingredients in the kit can be provided independently. For example, the kit may comprise one or more single-dose units of the anti-PD-1 antibody or the antigen binding fragment of the anti-PD-1 antibody (preferably toripalimab), and one or more single-dose units of the anti-folate metabolism anticancer drug (preferably, pemetrexed) and/or one or more single-dose units of the platinum anticancer drug (preferably, carboplatin). Preferably, the single-dose unit of the anti-PD-1 antibody or the antigen binding fragment of the anti-PD-1 antibody comprises the anti-PD-1 antibody or the antigen binding fragment of the anti-PD-1 antibody at about 120 mg to about 480 mg, such as 120 mg, 240 mg, 360 mg or 480 mg, preferably 240 mg or 360 mg; The single-dose unit of the anti-folate metabolism anticancer drug comprises the anti-folate metabolism anticancer drug at about 200 mg/m² to about 800 mg/m² of body surface area, for example 400 mg/m², 500 mg/m² or 600 mg/m² of body surface area, preferably 500 mg/m² of body surface area; The single-dose unit of the platinum anticancer drug comprises the platinum anticancer drug at about 200 mg/m² to about 800 mg/m² of body surface area, for example 400 mg/m², 500 mg/m² or 600 mg/m² of body surface area, preferably 500 mg/m² of body surface area.

In some embodiments, the kit comprises one or more single-dose units of the pharmaceutical composition described in any of the embodiments herein.

### Abbreviations

Throughout the specification and examples of the present invention, the following abbreviations are used:
BID one dose, 2 times a day
CDR complementarity determining region
DFS disease-free survival
FR framework region
IgG immunoglobulin G
IHC immunohistochemistry
OR overall response
s ORR objective response rate
OS overall survival
mOS mean overall survival
PD progressive disease
PFS progression-free survival
mPFS mean progression-free survival
PR partial response
CR complete response
SD stable disease
DLT dose-limiting toxicity
MTD maximum tolerated dose
AE adverse event
Q2W one dose every two weeks
QD one dose per day
CSD chronic sunshine duration
non-CSD non-chronic sunshine duration
IRC independent review committee
TRAE treatment-related adverse effect
SAE serious adverse effect
RO receptor occupancy
UC urothelial carcinoma
RCC renal cell carcinoma
MM metastatic melanoma
RECIST response evaluation criteria in solid tumors
irRECIST immune-related response evaluation criteria in solid tumors
DOR duration of response
MSI microsatellite instability

The present invention is further illustrated by the following examples, but the examples should not be construed as limiting the present invention. The contents of all references cited throughout the application are expressly incorporated herein by reference.

### EXAMPLES

### Example 1: Clinical Study of Anti-PD-1 Antibody Combined with Chemotherapy in the Treatment of Non-Small Cell Lung Cancer

Enrollment criteria: Eligible subjects must (1) be 18-75 years old, (2) have advanced or recurrent non-small cell lung cancer with EGFR-sensitive mutations, (3) fail the first-line EGFR-TKI treatment, (4) must have disease progression after receiving osimertinib treatment in the case of patients with T790M mutations, 5) have a ECOG score of 0 or 1, (6) have no history of autoimmune diseases, (7) have no previous anti-PD-1/or anti-PD-L1 immunotherapy.

Subjects must have evaluable lesions according to RECIST v 1.1 criteria, no combined small cell lung cancer or squamous cell carcinoma, no other mutations for targeted therapy, no previous systemic chemotherapy, and no long-term systemic immunosuppressive therapy.

From April 2018 to March 2019, there were 65 EGFR+NSCLC patients in 8 screening centers, and 40 patients were enrolled in this study. The median age of the subjects was 58 years (range: 19 to 73 years), including 21 (52.5%) women and 19 (47.5%) men. 23 patients (57.5%) had EGFR exon 19 deletion, while 17 patients (42.5%) had L858R mutation in exon 21. 20 patients (50.0%) received gefitinib as a first-line treatment, while 16 (40.0%) patients received icotinib and 4 (10.0%) patients received erlotinib as a first-line treatment. After the first-line TKI treatment, none of the 40 subjects had T790M drug-resistance mutations, but two (5%) subjects still received osimertinib as a second-line TKI treatment. 30 subjects participated in the study group using 360 mg toripalimab, and 10 subjects participated in the study group using 240 mg toripalimab. The demographic statistics of the enrolled subjects are shown in Table 1.

TABLE 1
Demographic statistics of the enrolled subjects

| Feature | Subjects (N = 40), n (%) | |
|---|---|---|
| Gender: Male/Female | 19 (47.5)/21 (52.5) | |
| Age, median (range) | 57.5 | (19-73) |
| Race: Asia | 40 | (100) |
| ECOG: 0/1 | 2 (5)/38 (95) | |
| Smoking history: Yes/No | 10 (25)/30 (75) | |
| EGFR mutation: Ex19 | 23 (57.5)/17 (42.5) | |
| deletion/L858R | | |
| CNS metastasis | 6 | (15) |
| Number of previous EGFR-TKI | 38 (95)/2 (5) | |
| treatments: 1/2 | | |
| PD-L1 tumor ratio score: | | |
| Negative | 19 | (47.5) |
| Positive | 21 | (52.5) |

Note:
*PD-L1 positive is defined as ≥1% of tumor cells expressing PD-L1 by JS311 IHC staining.
Test drugs: anti-PD-1 antibody terreplimumab (WO 2014206107), pemetrexed (commercially available) and carboplatin (commercially available).

In this study, the safety and clinical efficacy of two dosing regimens of toripalimab were compared. Enrolled subjects received 240 mg or 360 mg of toripalimab intravenously every 3 weeks until progressive disease or intolerable toxicity was confirmed. In the induction phase, subjects also needed to receive six periods of 500 mg/m² of pemetrexed plus carboplatin AUC 5 treatment, administrated intravenously once every three weeks. During the maintenance phase, subjects received 240 mg or 360 mg of toripalimab plus 500 mg/m² of pemetrexed.

According to the response evaluation criteria in solid tumors (RECIST) version 1.113 and irRECIST, assessment was made every 6 weeks. The treatment of a second progressive disease was not allowed in this study.

### Clinical Design:

This is a multi-center, non-blinded phase II clinical trial. This study was used to evaluate the safety and antitumor activity of an anti-PD-1 antibody combined with pemetrexed and carboplatin in the treatment of advanced or recurrent NSCLC with EGFR-sensitive mutations after failure of EGFR-TKI treatment.

### 1.1 Security Study:

As of Jun. 22, 2020, 39 of 40 patients (97.5%) have experienced treatment-related adverse effects (TRAEs). The most common (≥20%) TRAEs observed included 33 (82.5%) leukopenia, 28 (70.0%) neutropenia, 27 (67.5%) anemia, 21 (52.5%) elevated AST, 20 (50.0%) elevated ALT, 19 (47.5%) nausea, 19 (47.5%) thrombocytopenia, is cases (37.5%) decreased appetite, 11 (27.5%) constipation and 10 Cases (25.0%) debilitation (see Table 2). TRAEs with grade 3 and above occurred in 26 patients (65.0%), 4 patients (10%) were permanently discontinued with toripalimab due to TRAEs, while 15 patients (37.5%) were delayed in receiving toripalimab due to TRAEs. In the comparison of the 360 mg (n=30) and 240 mg (n=10) toripalimab groups, there was no significant difference in the incidence and severity of adverse effects (AEs).

TABLE 2
Common (>10%) treatment-related adverse effects (n = 40)

| | Total | | | |
|---|---|---|---|---|
| | incidence | | Incidence of | |
| AE parameters | (%) | | grade 3-5 (%) | |
| Patients with at least one TRAE | 39 | (97.5) | 26 | (65.0) |
| Leukopenia | 33 | (82.5) | 9 | (22.5) |
| Neutropenia | 28 | (70.0) | 16 | (40.0) |
| Anemia | 27 | (67.5) | 2 | (5.0) |
| Elevated aspartate aminotransferase | 21 | (52.5) | 2 | (5.0) |
| Elevated alanine aminotransferase | 20 | (50.0) | 2 | (5.0) |
| Nausea | 19 | (47.5) | 0 | |
| Thrombocytopenia | 19 | (47.5) | 4 | (10.0) |
| Decreased appetite | 15 | (37.5) | 1 | (2.5) |
| Constipation | 11 | (27.5) | 0 | |
| Debilitation | 10 | (25.0) | 0 | |
| Vomit | 7 | (17.5) | 0 | |
| Rash | 6 | (15.0) | 0 | |
| Urinary tract infection | 5 | (12.5) | 0 | |
| Elevated glutamyltransferase | 4 | (10.0) | 0 | |
| Influenza and other diseases | 4 | (10.0) | 0 | |
| Lung infection | 4 | (10.0) | 1 | (2.5) |
| Pyrexia | 4 | (10.0) | 0 | |
| Upper respiratory tract infection | 4 | (10.0) | 1 | (2.5) |

### 1.2 Antitumor Activity Study:

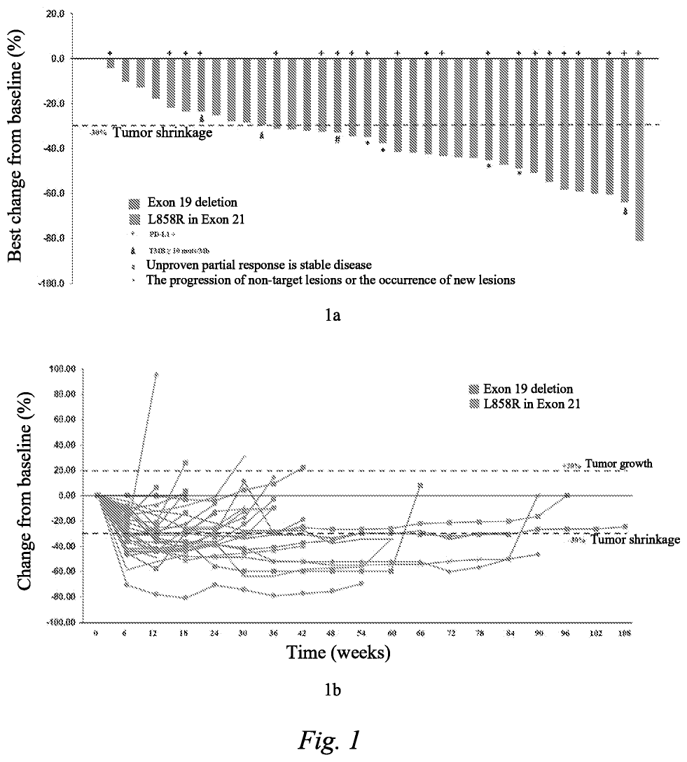

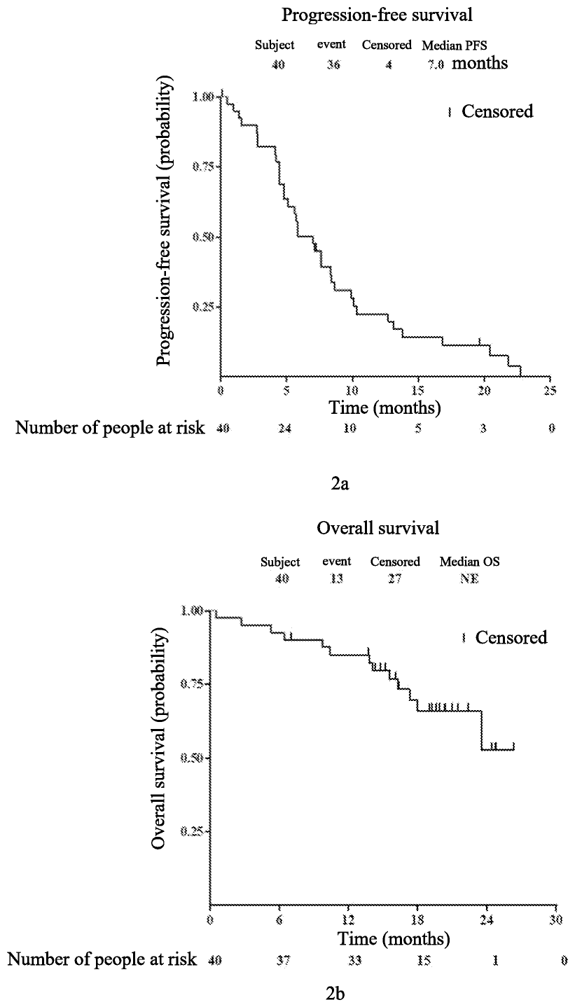

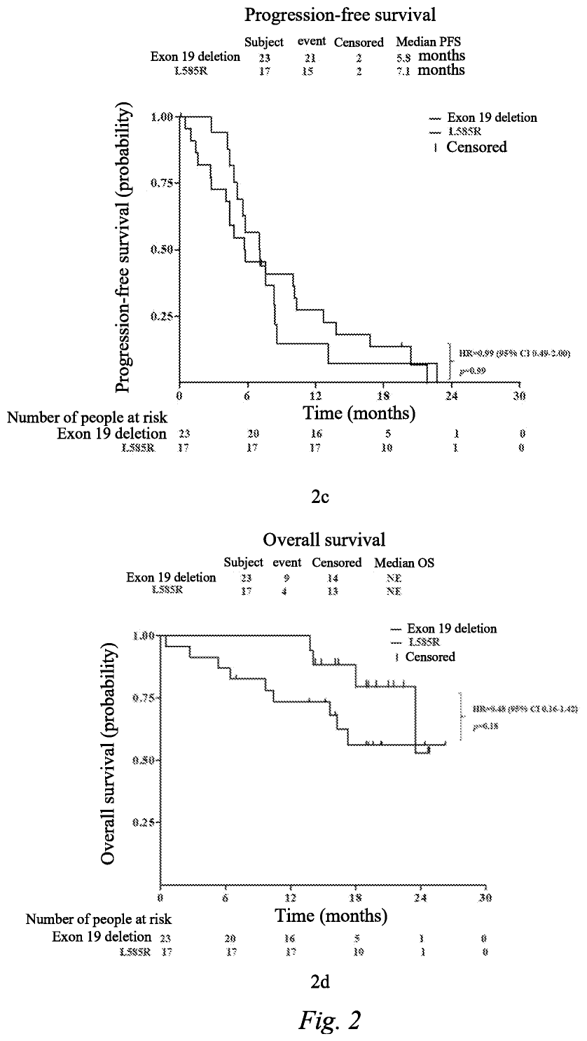

As of Jul. 10, 2020, among all 40 patients, the median follow-up time was 7.2 months, 13 patients (32.5%) died, the treatments for 22 patients (55.0%) were discontinued due to disease progression, and the treatments for 3 patients (7.5%) were discontinued due to AE, the study continued on 2 patients (5.0%). Among all 40 patients, 20 patients with partial remissions and 15 patients with stable disease were observed. The overall diagnosed ORR was 50% (95% CI: 33.8-66.2), and the DCR was 87.5% (95% CI: 73.2-95.8). The ORR at week 12 was 30.0%, 36 patients (90.0%) had a reduction in tumor size compared to baseline (FIG. 1a). The median DOR was 7.0 months (FIG. 1b). The median PFS was 7.0 months (95% CI: 4.8-8.4), the median OS was not reached (95% CI: 18.0-NE months) (FIGS. 2a and 2b). Using irRECIST criteria, the ORR of 21 PRs and 14 SDs was 52.5% (95% CI: 36.1-68.5), and the DCR was 87.5% (95% CI: 73.2-95.8).

Patients with EGFR L858R mutant (17 patients) had an ORR of 58.8%, and patients with exon 19 deletion (23 patients) had an ORR of 43.5%. Compared with patients with exon 19 deletion, patients with L858R mutation had longer PFS and overall survival (mOS NE vs NE, HR=0.48 [95% CI: 0.16-1.42], p=0.18), FIGS. 2c and 2d). Based on the first-line EGFR TKI treatment, the ORR of gefitinib (20 patients), icotinib (16 patients) and erlotinib (4 patients) was 35.0%, 62.5% and 75.0%, respectively. The ORR of each of the 360 mg toripalimab group (n=30) and 240 mg toripalimab group (n=10) was 50.0%.

### Example 2: Study on the Correlation Between Biomarkers and Clinical Efficacy 2.1 PD-L1 Expression in Tumors

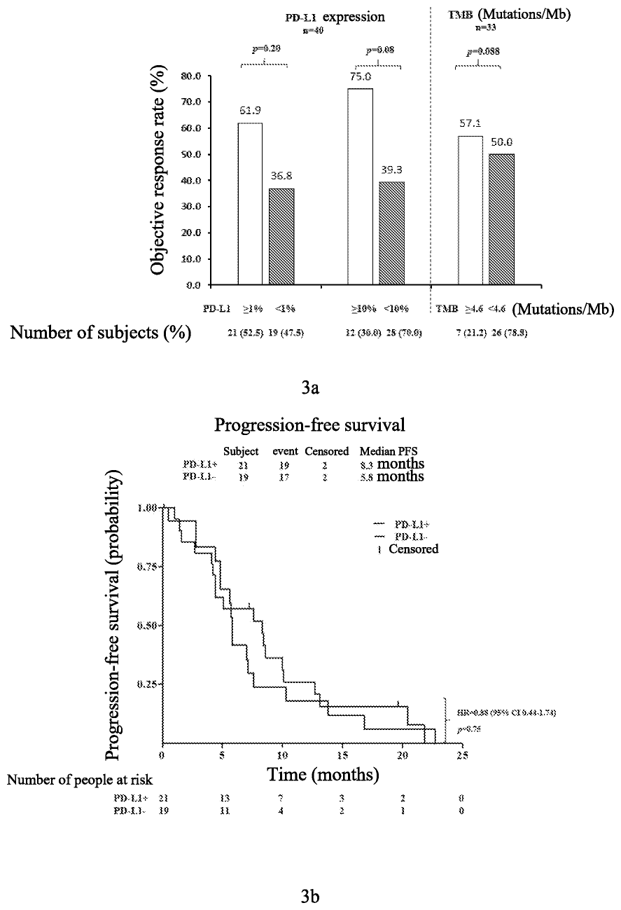

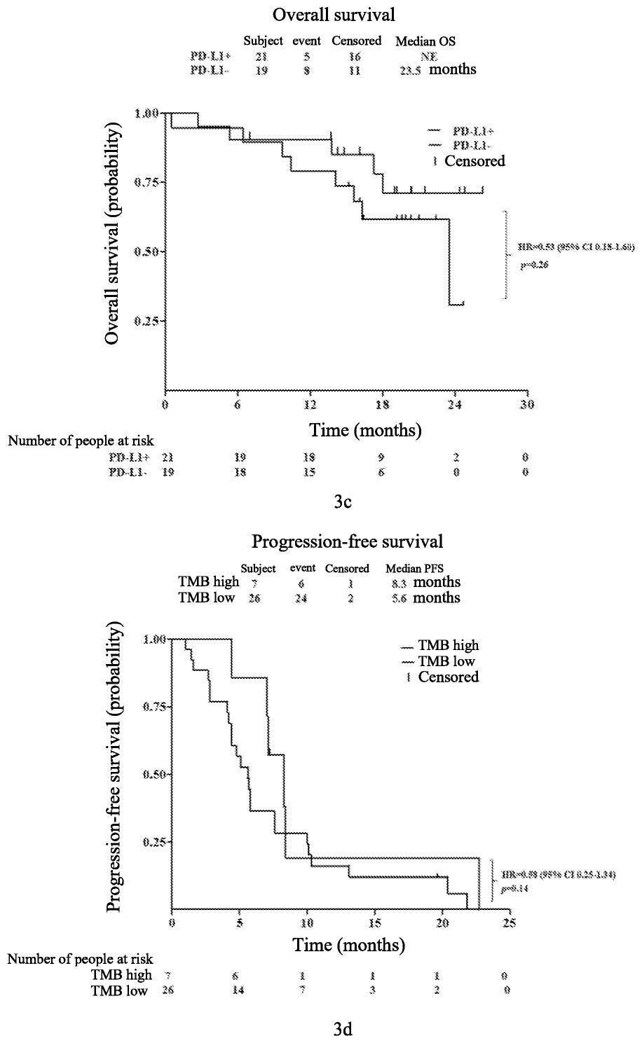

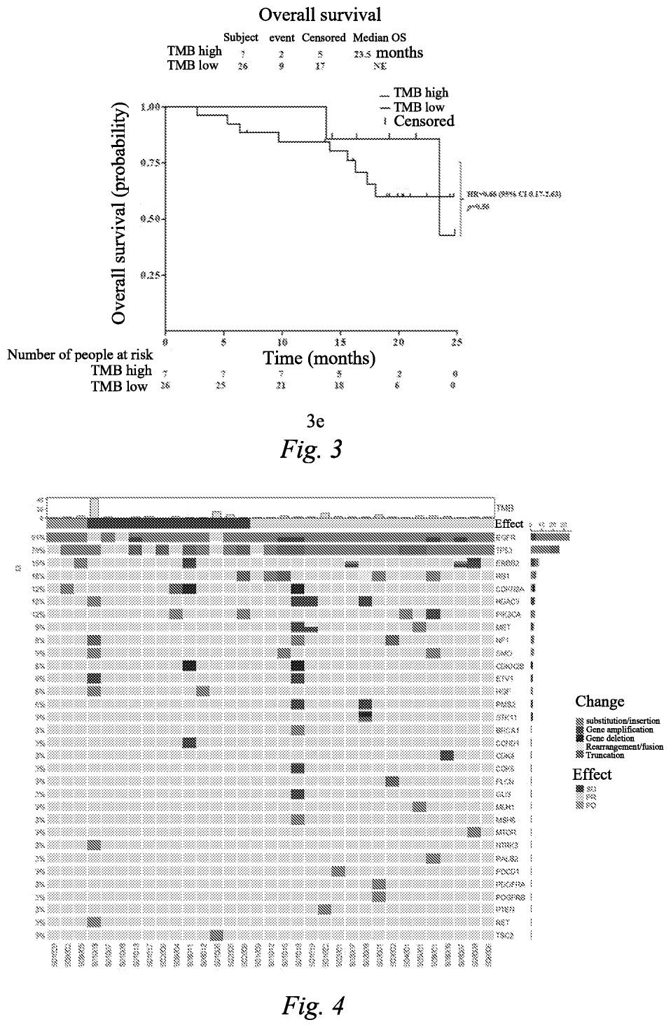

The PD-L1 expression status in tumor biopsy was confirmed by JS311 IHC staining, which showed that the staining results of 28-8, 22C3 and SP263 antibodies in NSCLC were similar. PD-L1+ (n=21) patients had higher ORR values than PD-L1− patients (n=19) (61.9% vs. 36.8%, p=0.20) (FIG. 3a). Among PD-L1+ patients, 12 patients (30.0%) were determined to have high PD-L1 expression (≥10%). In patients with PD-L1≥10%, the ORR difference was more pronounced (75.0% vs. 39.3%, p=0.08) (FIG. 3a). PD-L1+ patients had better PFS than PD-L1− patients (8.3 vs 5.8 months, HR=0.88 [95% CI: 0.44-1.74], p=0.71) and had better OS than PD-L1− patients (NE vs 23.5 months, HR=0.53) [95% CI: 0.18-1.60], p=0.26), but the difference was not statistically significant (FIGS. 3b and 3c).

### 2.2 Whole Exome Sequencing and Tumor Mutation Burden (TMB) Analysis

33 patients underwent tumor biopsy and matched peripheral blood was subjected to whole exome sequencing (WES). WES identified 7048 genetic mutations, comprising 3505 missense mutations, 84 gene deletions, 123 rearrangements, 119 splice site substitutions, 349 truncations and 2748 gene amplifications. In addition to EGFR mutations, the most common co-mutations/genomic changes comprise TP53 (79%), RB1 (18%), HER2 (15%), CDKN2A (12%), HDAC9 (12%). PIK3CA (12%), c-MET (9%), NF1 (9%) and SMO (9%) (FIG. 4). This study did not find a significant correlation between genomic changes and clinical efficacy. It was worth noting that two patients with c-MET amplification partially responded to the study treatment.

Tumor mutation burden (TMB) was determined by analyzing somatic mutations in the coding region of the human genome. The median of TMB in each group was 2.3 mutations (muts) per million base pairs (Mb). The TMB of 3 patients was greater than 10 muts/Mb (comprising 1 PR and 2 SD). According to the recommendations of Robert M. Samstein et al., this study chose the cut-off value (4.6 muts/Mb) of the top 20%. TMB to define TMB patients. Patients with TMB higher than 4.6 muts/Mb (n=7) had similar remission rates as patients with low TMB (n=26) (ORR 57.1% vs 50.0%) (FIG. 3a). The PFS value of patients with high TMB was better than that of patients with low TMB, but the difference was not statistically significant. The mPFS was 8.3 months and 5.6 months, respectively (HR=0.5895% CI: 0.25-1.34, p=0.20). The median OS of patients with high TMB was 23.5 months, while the mOS of patients with low TMB had not yet reached (FIGS. 3d and 3e).

1. A method of treating non-small cell lung cancer in a subject, comprising administering a subject in need thereof a combination of an anti-PD-1 antibody or an antigen binding fragment of the anti-PD-1 antibody and a cytotoxic anticancer drug; wherein the non-small cell lung cancer is a non-small cell lung cancer that has failed EGFR-TKI treatment.

2. The method according to claim 1, wherein preferably, the non-small cell lung cancer is advanced or recurrent non-small cell lung cancer that has failed EGFR-TKI treatment and has EGFR mutations.

3. The method according to claim 2, wherein the EGFR mutations are selected from exon 19 deletion and L858R mutation in exon 21.

4. The method according to claim 2, wherein the non-small cell lung cancer is a non-small cell lung cancer with PD-L1 expression ≥1% in immunohistochemical staining analysis of tumor tissue sections.

5. The method according to claim 1, wherein the anti-PD-1 antibody or the antigen binding fragment of the anti-PD-1 antibody comprises light chain complementarity determining regions with the amino acid sequences shown in SEQ ID NOs: 1, 2 and 3, and heavy chain complementarity determining regions with the amino acid sequences shown in SEQ ID NOs: 4, 5, and 6.

6. The method according to claim 5, wherein the anti-PD-1 antibody or the antigen binding fragment of the anti-PD-1 antibody comprises a light chain variable region with the amino acid sequence shown in SEQ ID NO: 7, and a heavy chain variable region with the amino acid sequence shown in SEQ ID NO: 8.

7. The method according to claim 6, wherein the anti-PD-1 antibody comprises a light chain with the amino acid sequence shown in SEQ ID NO: 9 and a heavy chain with the amino acid sequence shown in SEQ ID NO: 10.

8. The method according to claim 1, wherein the anti-PD-1 antibody is selected from one or more of nivolumab, pembrolizumab, toripalimab, sintilimab, camrelizumab, tislelizumab, and cemiplimab.

9. The method according to claim 1, wherein the cytotoxic anticancer drug is an anti-folate metabolism anticancer drug and/or a platinum anticancer drug.

10. The method according to claim 9, wherein the anti-folate metabolism anticancer drug is selected from methotrexate and pemetrexed; the platinum anticancer drug is selected from cisplatin, carboplatin and oxaliplatin.

11. The method according to claim 1, wherein the combination is a combination of toripalimab and pemetrexed, or a combination of toripalimab, pemetrexed and carboplatin.

12. The method according to claim 1, wherein,
(I) the single administration dose of the anti-PD-1 antibody or the antigen binding fragment of the anti-PD-1 antibody is about 0.1 mg/kg to about 10.0 mg/kg of individual body weight, or selected from a fixed dose of about 120 mg to about 480 mg;
(II) the cytotoxic anticancer drug is an anti-folate metabolism anticancer drug, wherein the single administration dose of the anti-folate metabolism anticancer drug is about 200 mg/m² to about 800 mg/m² of body surface area; and/or, the cytotoxic anticancer drug is an platinum anticancer drug, wherein the single administration dose of the platinum anticancer drug is about 200 mg/m² to about 800 mg/m² of body surface area.

13. The method according to claim 12, wherein,
(I) the dosing frequency of the anti-PD-1 antibody or the antigen binding fragment of the anti-PD-1 antibody is about once a week, once every two weeks, once every three weeks, once every four weeks, or once a month;
(II) the dosing frequency of the anti-folate metabolism anticancer drug is about once a week, once every two weeks, once every three weeks, once every four weeks or once a month;
(III) the dosing frequency of the platinum anticancer drug is about once a week, once every two weeks, once every three weeks, once every four weeks, or once a month.

14. The method according to claim 13, wherein,
(I) the anti-PD-1 antibody or the antigen binding fragment of the anti-PD-1 antibody has an administration dose of a fixed dose of 240 mg or 360 mg and administered once every three weeks;
(II) the anti-folate metabolism anticancer drug has an administration dose of 500 mg/m² of body surface area and administered once every three weeks;
(III) the platinum anticancer drug has an administration dose of 500 mg/m² of body surface area and administered once every three weeks.

15. The method according to claim 14, wherein the dosing period of the anti-PD-1 antibody or the antigen binding fragment of the anti-PD-1 antibody, the anti-folate metabolism anticancer drug and/or the platinum anticancer drug is one week, two weeks, three weeks, one month, two months, three months, four months, five months, six months or longer, optionally, each dosing period is the same or different, and the interval between each dosing period is the same or different.

16. The method according to claim 15, wherein the anti-PD-1 antibody or the antigen binding fragment of the anti-PD-1 antibody, the anti-folate metabolism anticancer drug and the platinum anticancer drug are administered in a liquid dosage form.

17. A pharmaceutical composition comprising an anti-PD-1 antibody or an antigen binding fragment of the anti-PD-1 antibody and a cytotoxic anticancer drug, wherein the cytotoxic anticancer drug is an anti-folate metabolism anticancer drug and/or a platinum anticancer drug.

18. The pharmaceutical composition according to claim 17, wherein,
the anti-PD-1 antibody or the antigen binding fragment of the anti-PD-1 antibody comprises light chain complementarity determining regions with the amino acid sequences shown in SEQ ID NOs: 1, 2 and 3, and heavy chain complementarity determining regions with the amino acid sequences shown in SEQ ID NOs: 4, 5, and 6;
the anti-folate metabolism anticancer drug is selected from methotrexate and pemetrexed;
the platinum anticancer drug is selected from cisplatin, carboplatin and oxaliplatin.

19.-21. (canceled)

22. The pharmaceutical composition according to claim 17, wherein,
the anticancer active ingredients in the pharmaceutical composition are toripalimab and pemetrexed, or the anticancer active ingredients in the pharmaceutical composition are toripalimab, pemetrexed and carboplatin.

23. A method for preventing or treating non-small cell lung cancer in a subject, comprising administering the pharmaceutical composition according to claim 17 to an individual in need, wherein
the cytotoxic anticancer drug is an anti-folate metabolism anticancer drug and/or a platinum anticancer drug; and
the anti-PD-1 antibody or the antigen binding fragment of the anti-PD-1 antibody comprises light chain complementarity determining regions with the amino acid sequences shown in SEQ ID NOs: 1, 2 and 3, and heavy chain complementarity determining regions with the amino acid sequences shown in SEQ ID NOs: 4, 5, and 6.

24. A kit, comprising:
(1) one or more single-dose units of the anti-PD-1 antibody or the antigen binding fragment of the anti-PD-1 antibody and one or more single-dose units of the cytotoxic anticancer drug; wherein
the anti-PD-1 antibody or the antigen binding fragment of the anti-PD-1 antibody comprises light chain complementarity determining regions with the amino acid sequences shown in SEQ ID NOs: 1, 2 and 3, and heavy chain complementarity determining regions with the amino acid sequences shown in SEQ ID NOs: 4, 5, and 6; and
the cytotoxic anticancer drug is an anti-folate metabolism anticancer drug and/or a platinum anticancer drug.
